(19) United States
(12) Patent Application Publication
(10) Pub. No.: US 2026/0073519 A1
(43) Pub. Date: Mar. 12, 2026

(54) DYNAMIC COMPUTED DYNAMIC COMPUTED TOMOGRAPHY IMAGING OF VASA VASORUM PERFUSION AND ANGIOGENESIS IN THE VASCULAR WALL

(71) Applicants: Morteza Naghavi, San Diego, CA (US); Chenyu Zhang, Rosemead, CA (US); Kyle Atlas, Huntington Beach, CA (US)

(72) Inventors: Morteza Naghavi, San Diego, CA (US); Chenyu Zhang, Rosemead, CA (US); Kyle Atlas, Huntington Beach, CA (US)

(21) Appl. No.: 19/390,629

(22) Filed: Nov. 16, 2025

Related U.S. Application Data
(63) Continuation-in-part of application No. 18/469,384, filed on Sep. 18, 2023.
(60) Provisional application No. 63/414,561, filed on Oct. 9, 2022.

(51) Int. Cl. G06T 7/00; A61B 6/42; A61B 6/50; G06N 3/044; G06N 3/08; G06T 7/12; G06T 17/00; G06V 10/764; G16H 15/00

(52) U.S. Cl. CPC G06T 7/0012; A61B 6/4241; A61B 6/504; G06N 3/044; G06N 3/08; G06T 7/12; G06T 17/00; G06V 10/764; G16H 15/00; G06T 2207/10081; G06T 2207/30104

(57) ABSTRACT

A method for quantitative mapping of vasa vasorum density within and adjacent to the coronary arterial wall using contrast-enhanced coronary CT angiography scans, including time-resolved perfusion, multi-energy material decomposition, and longitudinal functional monitoring of vasa vasorum dynamics.

### CROSS-REFERENCE

This application is Continuation-In-Part of the U.S. patent application Ser. No. 18/469,384 filed on Sep. 18, 2023, which claims priority to U.S. Provisional Patent Application No. 63/414,561, filed on Oct. 9, 2022, the entirety of which is incorporated herein by reference.

### FIELD OF THE INVENTION

The present invention relates to non-invasive imaging of vascular wall pathology, and more particularly to methods and systems for quantifying dynamic perfusion and angiogenic activity of the vasa vasorum (VV) in coronary and other arterial walls using time-resolved and spectral computed tomography (CT), optionally integrated with artificial intelligence (AI) analytics and multimodal data fusion.

### BACKGROUND

The parent invention introduced quantitative mapping of vasa vasorum density within and adjacent to the coronary arterial wall using CT-based micro-angiography. The present invention is an improvement of the parent invention that includes time-resolved perfusion, multi-energy material decomposition, and longitudinal functional monitoring of vasa vasorum dynamics.

Atherosclerosis is a chronic inflammatory disease of the arteries that is characterized by the formation of plaques on the inner walls of the arteries. These plaques can narrow or block the arteries, reducing blood flow to the heart, brain, and other organs. Atherosclerosis is the underlying cause of most heart attacks and strokes.

In the field of atherosclerotic plaque pathology, it has been known that proliferation of the vasa vasorum, the micro-vessels that supply oxygen and nutrients to the arterial wall, is associated with atherosclerosis and its complications. Atherosclerosis is very common and starts early in life. However, its complications, including heart attacks and strokes, which are the leading causes of death worldwide, occur later in life when atherosclerotic plaques become vulnerable to atherothrombotic events.

Vulnerable atherosclerotic plaques are plaques that are more likely to rupture and or cause a blood clot. These plaques are typically characterized by a large amount of lipids (fats) and a thin fibrous cap. When a plaque ruptures, it can trigger the formation of a blood clot, which can block the artery and lead to a heart attack or stroke. For this to occur clinically, and to result in fatal outcomes, usually other factors including blood thrombogenesity and myocardia vulnerability to arrythmia are required.

Inflammation (which is a broadly defined word to represent the reaction of body's immune system to defend against unexpected stimuli) plays a key role not only in the development of atherosclerosis but also in its complications. An active inflammation site is like an active battleground where immune cells constantly attack the unknown agents with cellular and humoral pathways. To maintain and succeed in this fight, the immune cells require constant delivery of blood that carries oxygen and nutrients. Almost always new blood vessels are needed much like new roads are needed to deliver foods and ammunition to a battlefront, such roads are microvessels and such a phenomenon is called angiogenesis, and such angiogenesis in and around the arteries become an extension of the vasa vasorum network which are microvessels feeding the vessel walls. These newly formed vessels are often lose and leaky hence can lead to extravasation of contrast agents inside an atherosclerosis plaque which is sometimes referred to as a “blush sign” in an X-ray image after injection of the an X-ray dye. Active immune cells like macrophages are like massive bodybuilders that engulf foreign bodies and oxidized lipid molecules hence need much more fuel than ordinary cells in the arterial wall hence the need for more blood supply and more vasa vasorum. Imaging these excess microvessels and their blood circulation using X-ray dye mixed blood under CT scans is the focus on this invention.

While it has been long perceived that imaging inflammation can be a useful metric in evaluating the risk of atherosclerosis, it has been difficult to assess using non-invasive means. Recent reports by investigators who claim imaging periadventitial fat is a reliable way of imaging coronary inflammation meets the skepticism of experts. The investigators insist that their technique is able to measure lipolysis by measuring reduction of fat signals around atherosclerotic plaques. This invention takes a contrary view and focused on measuring the increased Hounsfield density in and around the atherosclerotic plaques and coronary wall due to increase density of vasa vasorum and resulting HU enhancement by X-ray contrast agents (X-ray dye). Numerous pathology and CT imaging studies have clearly demonstrated increased fat around coronary arteries including epicardial and pericardial fat is associated with poor outcomes. Therefore, the notion of fat reduction contrasts such a large body of evidence. Contrary to fat attenuation hypothesis, the vasa vasrom density approach is in line with the common understanding of what goes on in body's sites of inflammation.

Furthermore, existing technologies, such as Caristo Diagnostics'perivascular fat attenuation index (FAI), rely on static attenuation changes in perivascular adipose tissue, not on intravascular perfusion or micro-angiogenesis. They do not capture temporal enhancement kinetics, contrast wash-in/wash-out patterns, or response to therapy—all of which are critical indicators of vascular wall inflammation.

Therefore, practical imaging methods and systems for detection of increased vasa vasorum density and monitoring changes in vasa vasorum density are urgently needed.

### SUMMARY OF INVENTION

The present invention provides methods and systems for dynamic imaging, quantification, and AI analysis of vasa vasorum perfusion to assess inflammatory and angiogenic activity in the vascular wall.

According to an aspect of the present invention, there is provided a method for sequential CT imaging captures changes in enhancement within the vascular wall and adventitia, generating VV perfusion curves that represent blood-flow kinetics, and dual-energy or photon-counting CT separates material components (iodine, calcium, lipid) to isolate microvascular contrast signals.

According to another aspect of the present invention, an AI-Derived Temporal Modeling that predicts and classifies angiogenesis progression using temporal enhancement patterns, comprising: longitudinal comparison of VV density and perfusion between baseline and follow-up scans assesses treatment efficacy or disease progression, integration with MRI, PET, or OCT enables multimodal confirmation of vascular inflammation and angiogenesis, combination of VV metrics with systemic biomarkers (LDL, hsCRP, cytokines) to predict future cardiovascular events, and AI-enabled workstation modules automate wall segmentation, perfusion computation, and clinical reporting.

### BRIEF DESCRIPTION OF THE DRAWINGS

FIG. 1 illustrates the anatomy of an artery and the three layers of an arterial wall. Vasa vasorum are illustrated as tiny vessels surrounded and penetrating the arterial wall mostly from the outside (adventitial layer) but also from the inside (endothelial or luminal side).

FIG. 2 illustrates prior art vasa vasorum imaging using microbubbles contrast-enhanced intravascular ultrasound (IVUS) subtraction analysis that only shows the microvessels containing microbubbles.

FIG. 3 illustrates vasa vasorum in contrast-enhanced intravascular ultrasound, contrast-enhanced microCT, and pathology slides.

FIG. 4 illustrates a demonstration of peri-arterial (adventitial and peri-adventitial) Hounsfield Unit heterogeneity caused by the network of vasa vasorums and CT contrast agent (X-ray dye) circulating inside the vasa vasorum. High intensity sports resemble larger vasa vasorums with higher amounts of blood containing X-ray dye.

FIG. 5 illustrates normal vasa vasorum (low VV density) and vasa vasorum in an atherosclerotic arterial wall (high VV density).

FIG. 6 illustrates a flowchart of a method used in embodiments for evaluating risk and recommending the next diagnostic or therapeutic step based on the vasa vasorum density observed.

FIG. 7 illustrates a cross-section of an arterial wall depicting the distribution of vasa vasorum. The figure contrasts a stable arterial wall with minimal vasa vasorum versus an inflamed arterial wall exhibiting extensive neovascularization penetrating the media and adventitia. The depiction highlights how increased vasa vasorum density corresponds to vascular inflammation and early angiogenesis.

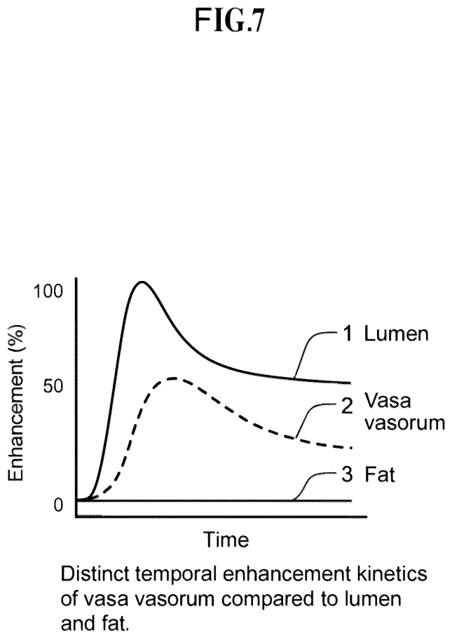

FIG. 8 is a comparison between a fat-attenuation-based map (e.g., perivascular fat attenuation index), which applies Hounsfield Unit filtering to adipose tissue surrounding the vessel, and a vasa-vasorum-based perfusion map generated using the disclosed CT methods without HU filtering, enabling direct visualization of blood-flow patterns within the arterial wall. The figure demonstrates how the disclosed approach resolves wall-level perfusion, which is not captured by fat-only metrics.

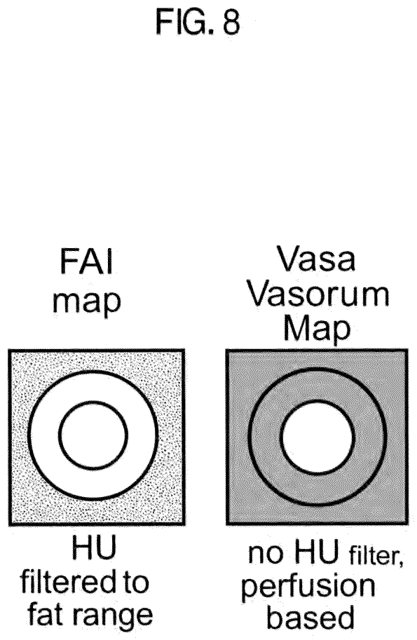

FIG. 9 depicts two cross-sectional arterial wall illustrations of an inflamed coronary artery with dense, branching vasa vasorum penetrating inward, and a stable plaque with only a minimal number of small vasa vasorum branches. The figure visually emphasizes how perfusion-derived vascularity differs between inflamed and stable atherosclerotic lesions.

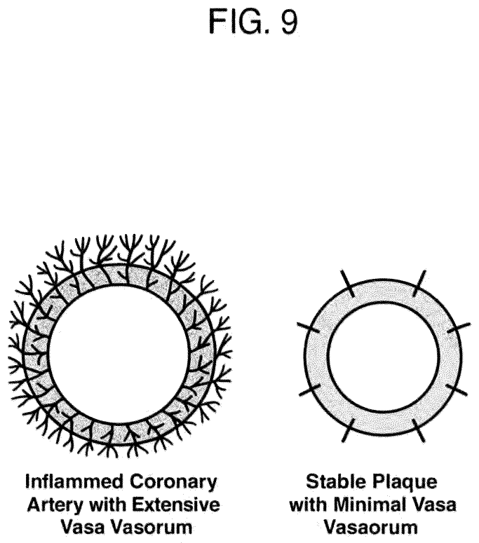

FIG. 10 illustrates multiple sequential CT images acquired after administration of contrast, showing time-resolved enhancement of the vascular wall. The series represents dynamic perfusion, where each temporal frame contributes to per-voxel time-density curves (TDCs) used to quantify vasa vasorum flow, peak enhancement, wash-in, and wash-out behavior.

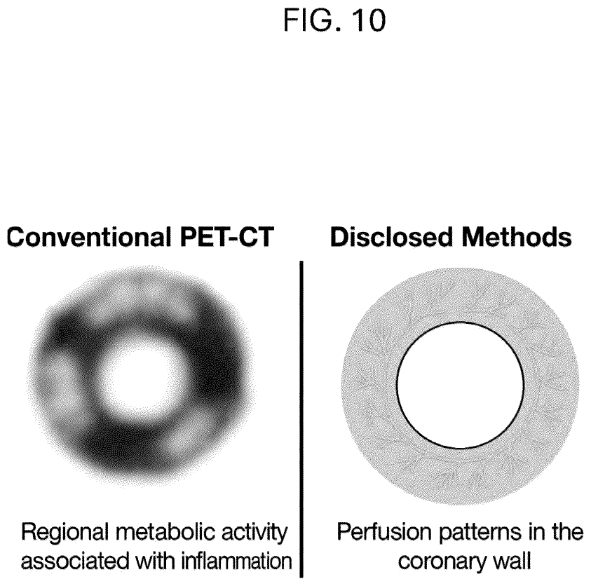

FIG. 11 depicts an example of dual-energy or photon-counting CT material decomposition used to isolate iodine maps within the arterial wall. The iodine map is used as a direct surrogate for microvascular blood volume, enabling measurement of perfusion attributable to vasa vasorum. The figure demonstrates how spectral CT separates iodine from calcium and soft tissue to better characterize angiogenesis.

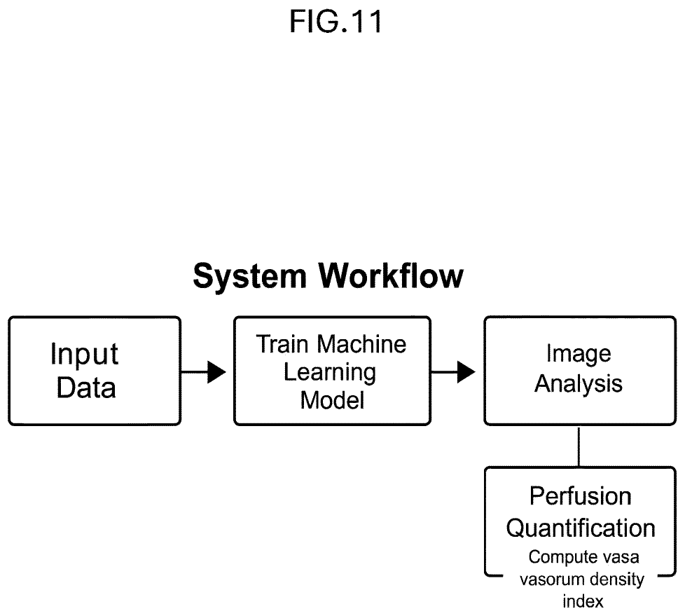

### DETAILED DESCRIPTIONS

Detection of increased vasa vasorum during contrast-enhanced computed tomography (CT) imaging is indicative of coronary artery disease.

Hence the new invention utilizes contrast enhanced coronary CT scans to measure vasa vasorum density in and around the coronary arteries. The principle foundations of this approach is based on the well-established knowledge that inflammation in particular chronic inflammation such as atherosclerotic coronary artery disease and other forms of chronic vasculitis result in excess proliferation of vasa vasorum, the tiny vessels that feed the arteries. The more inflammation the more traffic of blood flow to the area which requires higher density of microvasculature.

In one embodiment, a CT scan can be performed, with and without contrast agent, starting with non-contrast as the screening step, and using contrast-enhanced coronary angiography for a selected population, with interpretation of the results of both automated powered by artificial intelligence (AI).

In another embodiment, two or more CT scans of the same vascular segment are acquired—either within seconds (dynamic perfusion) or months (longitudinal therapy response). Each scan captures contrast kinetics or baseline-to-enhanced transitions, enabling voxel-level perfusion analysis of the vasa vasorum.

In another embodiment, two or more CT scans are acquired of the same vascular segment—either within seconds (dynamic perfusion) or months (longitudinal therapy response). Each scan captures contrast kinetics or baseline-to-enhanced transitions, enabling voxel-level perfusion analysis of the vasa vasorum.

In another embodiment, time-density curves (TDC) or contrast enhancement ratios for voxels within the vascular wall is computed to derive parameters such as peak enhancement, wash-in slope, wash-out rate, time-to-peak, and area-under-curve (AUC) for each voxel.

In another embodiment, in dual-energy CT, data are decomposed into iodine, calcium, soft-tissue maps, and iodine-based perfusion values are extracted within the wall to directly measure blood volume fraction and distinguish active angiogenesis from fibrosis or lipid content.

Another embodiment uses deep neural networks or Machine Learning Model (CNNs, RNNs, or transformers) associated with a server to train on dynamic CT sequences to classify each wall segment such as Stable, Inflamed, or Actively Angiogenic, and compute a Vasa Vasorum Perfusion Index (VVPI) summarizing perfusion kinetics and temporal heterogeneity. Perfusion parameters between baseline and follow-up studies are then compared to evaluate and determine effectiveness of anti-inflammatory or lipid-lowering therapy, and natural progression of subclinical atherosclerosis.

In another embodiment, the method co-registers VV maps with MRI (T1/T2 mapping), PET (\hat{ }18F-FDG or \hat{ }68Ga-DOTATATE), or intravascular imaging (IVUS/OCT) to confirm inflammation and angiogenesis, and to integrate VV perfusion metrics into clinical decision systems to generate automated reports and recommendations for further testing or therapy adjustments.

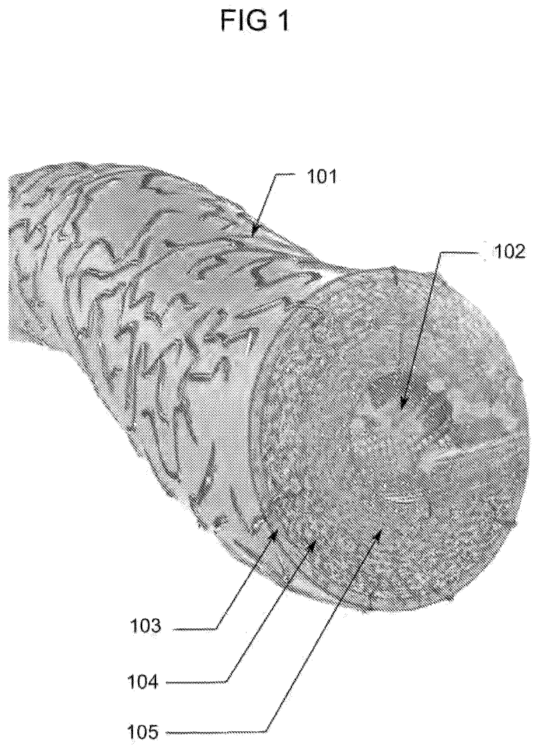

FIG. 1 illustrates the anatomy of an artery and the three layers of an arterial wall. Vasa vasorum are illustrated as tiny vessels surrounded and penetrating the arterial wall mostly from the outside (adventitial layer) but also from the inside (endothelial or luminal side). Vasa vasourm 101 are the vessels situated on the wall of the artery. Oxygenated blood flows through lumen 102 to supply nutrients and oxygen to cardiac muscle. Tunica adventitia 103 is the outer layer of the artery wall. Tunica media 104 is the middle layer of the artery wall. Tunica intima 105 is the inner layer of the artery wall.

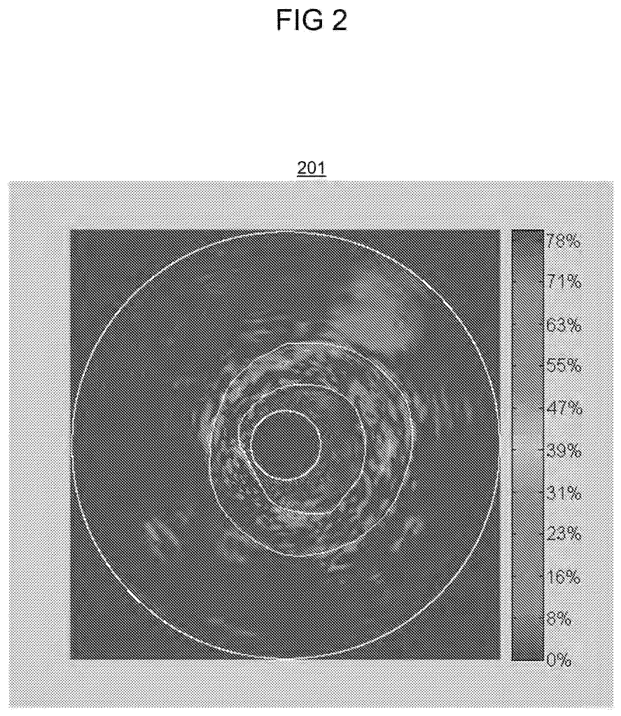

FIG. 2 illustrates prior art vasa vasorum imaging using microbubbles contrast enhanced intravascular ultrasound (IVUS) subtraction analysis that only shows the microvessels containing microbubbles.

Still in FIGS. 2, 201 refers to prior art vasa vasorum imaging using microbubbles contrast-enhanced intravascular ultrasound (IVUS).

Coronary vasa vasorum imaging using microbubbles and intravascular ultrasound (IVUS) imaging devices is known. However, IVUS is a highly invasive procedure that requires a catheterization laboratory (like an OR) and penetrating patient's skin to access femoral or radial arteries and threading the IVUS catheter to the aorta and from the root of aorta into each of the major branches of coronary arteries. This is not only invasive but also expensive and ethically unjustifiable for most patients who could benefit from such information.

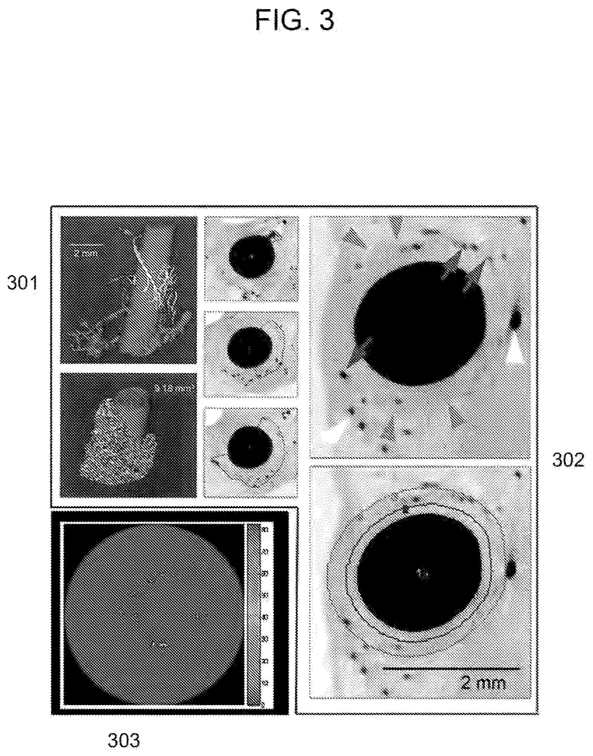

FIG. 3 illustrates vasa vasorum in contrast-enhanced intravascular ultrasound, contrast-enhanced microCT, and pathology slides.

Contrast-enhanced microCT image 301 is obtained using Micro-CT. Micro-CT scanning is X-ray imaging in 3D, using the same method as medical CT (or “CAT”) scans, but micro-CT is on a much smaller scale with greatly increased resolution. Pathology slides 302 have been prepared by a pathologist slicing the tissue block containing vasa vasorum into very thin layers that are placed on a glass slide and examined under a microscope. Contrast enhanced intravascular ultrasound (IVUS) image 303 is a prior art image as described with reference to FIG. 2.

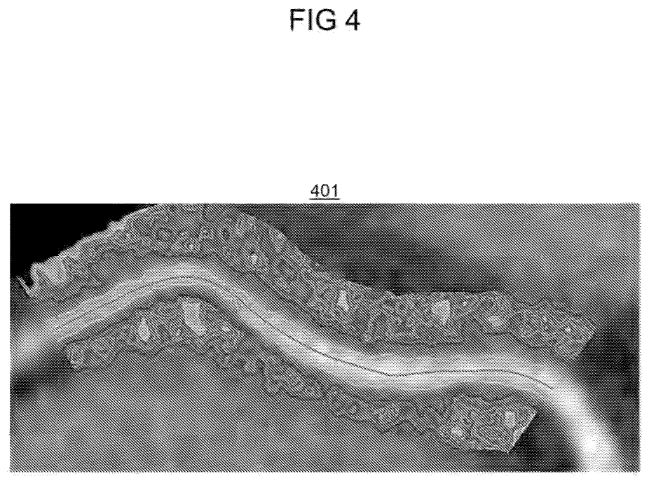

FIG. 4 illustrates a demonstration of peri-arterial (adventitial and peri-adventitial) Hounsfield Unit heterogeneity caused by the network of vasa vasorums and CT contrast agent (X-ray dye) circulating inside the vasa vasorum. High intensity sports resemble larger vasa vasorums with higher amounts of blood containing X-ray dye.

Image 401 in FIG. 4 illustrates a demonstration of Arterial Wall and Peri-Arterial Hounsfield unit heterogeneity caused by vasa vasorum density and CT contrast agent circulating inside the vasa vasorum.

It can be observed that there is heterogeneity in the attenuation around the coronary arteries depicted in FIG. 4, as measured in Hounsfield units (HU), where Hounsfield units are defined as the attenuation value of the X-ray beam in a given voxel, minus the attenuation of water, divided by the attenuation of water, multiplied by 1000.

The reason for heterogeneity in the HU density of the surrounding of the wall is the vasa vasorum surrounding the wall. The vasa vasorum is a network of vessels and as such has spaces between the vessels where attenuation is less. If the attenuation was being caused by adipose tissue, the heterogeneity observed would not be present. The HU density of the actual wall of the coronary arteries is also affected by changes in the density of VV.

The more inflammation, the higher the VV density, the more contrast agent circulating inside VV in & around the coronary walls, the higher the HU density in & around the coronary wall. In contrast, the more intensive treatment, the less inflammation, the less VV density, the less contrast agent circulating inside VV in & around the coronary walls, the less the HU density in & around the coronary wall.

The change is HU density around the coronary walls observed in a second CT performed without administering additional contrast agent some time after a first contrast CT of VV of a coronary artery proves that the vasa vasorum is causing the attenuation observed in the first contrast CT scan. The contrast agent circulates in the vessels of the VV, and as the concentration of the contrast agent declines (because the contrast agent is eliminated), the observed HU density decreases.

The alternative hypothesis that the HU density around the coronary walls observed on a contrast coronary CT is due to the density of fat around the walls is mistaken because the HU attenuation due to the density of fat would be the same on a second contrast CT taken after some time and without administration of additional contrast agent, and in fact HU attenuation declines on the repeat CT.

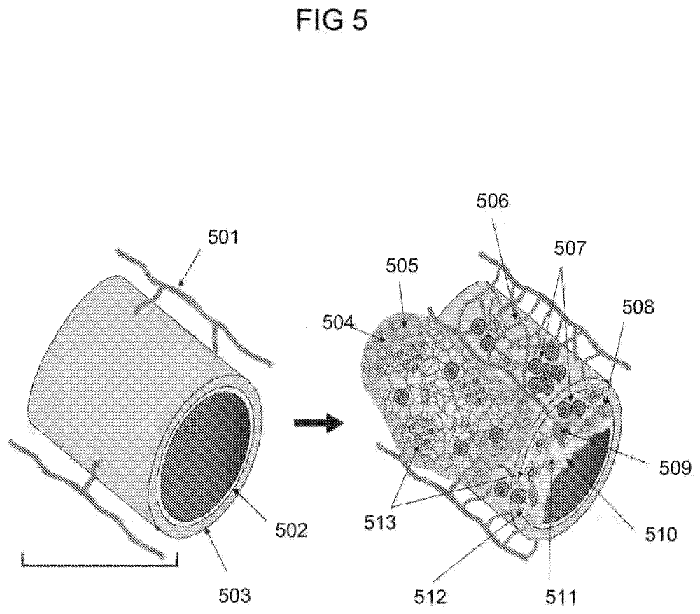

FIG. 5 illustrates normal vasa vasorum (low VV density) and vasa vasorum in an atherosclerotic arterial wall (high VV density).

On the left of FIG. 5, vasa vasorum 501 services a normal artery with intima 502 and media 503. This is a normal artery without atherosclerosis present.

On the right of FIG. 5, proliferated vasa vasorum 506 serves an artery showing signs of atherosclerosis. Inflammatory cells 507 and smooth muscle cells 508 contribute towards a narrowing of the atherosclerotic artery. Intraplaque hemorrhage 509 is blood pooled in the artery wall. Athersclerotic plaque 510 narrows the artery wall and causes risks associated with cardiovascular disease. Necrotic core 511 is a hypocellular region containing remnants of dead cells. Invasion of vasa vasorum into the plaque 512 is the blood vessel network serving the plaque. Macrophages 513 are recruited to the necrotic core 511. Perivascular adipose tissue 504 is fat surrounding the artery. Capillaries in adipose tissue 505 are the blood vessels which service the perivascular adipose tissue.

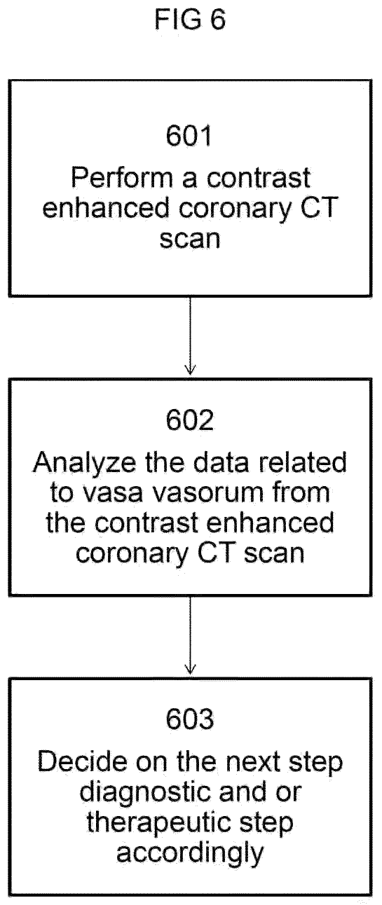

FIG. 6 illustrates a flowchart of a method used in embodiments for evaluating risk and recommending the next diagnostic or therapeutic step based on the vasa vasorum density observed.

Step 601 is performing a contrast enhanced coronary CT scan to measure attenuation related to vasa vasorum density in and around the coronary arteries of a patient. For example, data could be gathered along a length of one or more of left main coronary artery, left anterior descending coronary artery, left circumflex coronary artery, and right coronary artery.

Step 602 is analyzing the data from the contrast enhanced coronary CT scan to determine a metric related to the density of the vasa vasorum in and around the coronary arteries of the patient. Analysis of the data could be done using artificial intelligence, for example, on a deep neural network trained with many images of vasa vasorum.

Step 603 is deciding whether and what therapy and/or a diagnostic test to administer to the patient to treat or prevent cardiovascular disease based at least in part on the metric related to the density of the vasa vasorum in and around the coronary arteries of the patient. Treatment for atherosclerosis may include lifestyle changes, medicine, and surgery, for example.

References regarding vasa vasorum and atherosclerosis include the following articles.

Yan A, Gotlieb AI. The microenvironment of the atheroma expresses phenotypes of plaque instability. Cardiovasc Pathol. 2023 Aug. 16; 67:107572. doi:10.1016/j.carpath.2023.107572. Epub ahead of print. PMID: 37595697. https://pubmed.ncbi.nlm.nih.gov/37595697/

Chen D, Zhao Z, Liu P, Liu X, Wang X, Ren Q, Chang B. Adventitial Vasa Vasorum Neovascularization in Femoral Artery of Type 2 Diabetic Patients with Macroangiopathy Is Associated with Macrophages and Lymphocytes as well as the Occurrence of Cardiovascular Events. Thromb Haemost. 2023 Apr. 10. doi: 10.1055/s-0043-1768162. Epub ahead of print. PMID: 37037199. https://pubmed.ncbi.nlm.nih.gov/37037199/

Hillock-Watling C, Gotlieb AI. The pathobiology of perivascular adipose tissue (PVAT), the fourth layer of the blood vessel wall. Cardiovasc Pathol. 2022 November December; 61:107459. doi: 10.1016/j.carpath.2022.107459. Epub 2022 Jul. 28. PMID: 35907442. https://pubmed.ncbi.nlm.nih.gov/35907442/

Guggenberger KV, Torre GD, Ludwig U, Vogel P, Weng AM, Vogt ML, Fröhlich M, Schmalzing M, Raithel E, Forman C, Urbach H, Meckel S, Bley TA. Vasa vasorum of proximal cerebral arteries after dural crossing—potential imaging confounder in diagnosing intracranial vasculitis in elderly subjects on black-blood MRI. Eur Radiol. 2022 February; 32(2):1276-1284. doi: 10.1007/s00330-021-08181-5. Epub 2021 Aug. 4. PMID: 34347156; PMCID: PMC8795054. https://pubmed.ncbi.nlm.nih.gov/34347156/

Li M, Qi Z, Zhang J, Zhu K, Wang Y. Effect and Mechanism of Si-Miao-Yong-An on Vasa Vasorum Remodeling in ApoE−/− Mice with Atherosclerosis Vulnerable Plague. Front Pharmacol. 2021 Apr. 14; 12:634611. doi: 10.3389/fphar.2021.634611. PMID: 33935723; PMCID: PMC8080061. https://pubmed.ncbi.nlm.nih.gov/33935723/ Ito H, Wakatsuki T, Yamaguchi K, Fukuda D, Kawabata Y, Matsuura T, Kusunose K, Ise T, Tobiume T, Yagi S, Yamada H, Soeki T, Tsuruo Y, Sata M. Atherosclerotic Coronary Plaque Is Associated With Adventitial Vasa Vasorum and Local Inflammation in Adjacent Epicardial Adipose Tissue in Fresh Cadavers. Circ J. 2020 Apr. 24; 84(5): 769-775. doi: 10.1253/circj.CJ-19-0914. Epub 2020 Apr. 10. PMID: 32281556. https://pubmed.ncbi.nlm.nih.gov/32281556/

Cattaneo M, Sun J, Staub D, Xu D, Gallino JM, Santini P, Porretta AP, Yuan C, Balu N, Arnold M, Froio A, Limoni C, Wyttenbach R, Gallino A. Imaging of Carotid Plaque Neovascularization by Contrast-Enhanced Ultrasound and Dynamic Contrast-Enhanced Magnetic Resonance Imaging. Cerebrovasc Dis. 2019; 48(3-6): 140-148. doi: 10.1159/000504042. Epub 2019 Oct. 29. PMID: 31661690. https://pubmed.ncbi.nlm.nih.gov/31661690/

Quan K, Song J, Yang Z, Wang D, An Q, Huang L, Liu P, Li P, Tian Y, Zhou L, Zhu W. Validation of Wall Enhancement as a New Imaging Biomarker of Unruptured Cerebral Aneurysm. Stroke. 2019 June; 50(6):1570-1573. doi: 10.1161/STROKEAHA.118.024195. Epub 2019 Apr. 30. PMID: 31035900. https://pubmed.ncbi.nlm.nih.gov/31035900/

Papaioannou TG, Vavuranakis M, Androulakis A, Lazaros G, Kakadiaris I, Vlaseros I, Naghavi M, Kallikazaros I, Stefanadis C. In-vivo imaging of carotid plaque neoangiogenesis with contrast-enhanced harmonic ultrasound. Int J Cardiol. 2009 May 29; 134(3):e110-2. doi: 10.1016/j.ijcard.2008.01.020. Epub 2008 May 20. PMID: 18495267. https://pubmed.ncbi.nlm.nih.gov/18495267/

Having thus described a few particular embodiments of the invention, various alterations, modifications, and improvements will readily occur to those skilled in the art. Such alterations, modifications, and improvements are intended to be part of this description though not expressly stated herein, and are intended to be within the spirit and scope of the invention. Accordingly, the foregoing description is by way of example only, and not limiting. None of the descriptions in the specification are intended to be imported into the claims, and nothing in the specification should be construed to limit any of the claims below.

We claim:

1. A method for detecting vascular inflammation and angiogenesis in a vascular wall using computed tomography (CT), comprising:
training a Machine Learning Model (MLM) to train on a paired non-contrast and contrast enhanced CT scans to segment the vessel wall;
acquiring at least two CT datasets of the same vascular segment at different time points following administration of contrast;
quantifying time-dependent enhancement within the wall to generate a plurality of voxel-level perfusion curves representing microvascular blood flow;
computing a vasa vasorum density index (VVDI) and vasa vasorum perfusion index (VVPI), based on the intensity values and time-dependent intensity changes from one or more temporal parameters including peak enhancement, time-to-peak, wash-in rate, and area-under-curve for each voxel within and surrounding the segmented wall;
displaying the VVDI and VVPI parameters in color-coded parametric overlay on the segmented wall and surrounding; and
comparing VVDI and VVPI parameters between baseline and follow-up studies, to determine effectiveness of anti-inflammatory or lipid therapy, and natural progression of subclinical atherosclerosis.

2. The method of claim 1, wherein the plurality of voxel-level perfusion curves is generated from dynamic CT frames acquired during a single contrast injection.

3. The method of claim 1, wherein VVDI and VVPI parameters are normalized to an arterial input function (AIF) derived from lumen enhancement to minimize the blooming effect.

4. The method of claim 1, wherein VVDI and VVPI parameters are expressed as a ratio of wall enhancement to blood pool enhancement.

5. The method of claim 1, wherein the MLM comprises a recurrent neural network trained to model temporal dependencies in perfusion data.

6. The method of claim 1, wherein changes in VVDI and VVPI parameters between scans are used to assess response to anti-inflammatory or lipid-lowering therapy.

7. The method of claim 1, further comprising a step of co-registering the vasa vasorum perfusion map with MRI or PET configured to validate regions of inflammation.

8. The method of claim 1, wherein changes in perfusion parameters between scans are used to assess response to anti-inflammatory or lipid-lowering therapy.

9. The method of claim 2, wherein VVDI and VVPI are depicted as color-coded parametric overlays on the segmented wall and surrounding.

10. The method of claim 1, further comprising a step of recommending next diagnostic or therapeutic step based on VVDI, VVPI, and their trends.

11. A method for characterizing vasa vasorum perfusion using dual-energy or photon-counting CT, comprising:
training a Machine Learning Model (MLM) to train on a paired non-contrast and contrast enhanced CT scans to segment the vessel wall;
acquiring spectral CT images of the vessel wall;
performing material decomposition to isolate iodine concentration maps inside and surrounding the wall;
quantifying iodine-based enhancement as a proxy for vasa vasorum blood volume;
generating a three-dimensional perfusion density map; and
classifying vascular segments as inflamed, stable, or fibrotic based on iodine distribution.

12. The method of claim 11, wherein a photon-counting CT is used to simultaneously quantify iodine and calcium for differentiating active plaque from calcified plaque.

13. A computer-implemented method for monitoring progression or regression of vascular inflammation, comprising:
training a Machine Learning Model (MLM) to train on a paired non-contrast and contrast enhanced CT scans to segment the vessel wall;
obtaining baseline and follow-up CT scans of the same patient;
extracting vasa vasorum perfusion features from both scans;
feeding the temporal features into a trained machine learning model (MLM) configured to output a vasa vasorum activity index (VVAI); and
outputting a clinical recommendation output regarding therapeutic response or risk of cardiovascular event.

14. The method of claim 13, wherein the MLM comprises a recurrent neural network trained to model temporal dependencies in perfusion data.

15. The method of claim 13, wherein the clinical recommendation output comprises a categorical classification of “progressing,” “stable,” or “regressing” angiogenesis.

16. The method of claim 15, wherein the clinical recommendation output is integrated into a clinical decision-support system for generating a report.
